(19) United States
(12) Patent Application Publication
(10) Pub. No.: US 2016/0089325 A1
(43) Pub. Date: Mar. 31, 2016

(54) SOAP COMPOSITION

(71) Applicant: IINN, INC., Flint, MI (US)

(72) Inventor: Jeremiah R. Perkins, Corunna, MI (US)

(73) Assignee: INSIGHT INSTITUTE OF NEUROSURGERY & NEUROSCIENCE, Flint, MI (US)

(21) Appl. No.: 14/787,916

(22) PCT Filed: Apr. 29, 2014
(86) PCT No.: PCT/US14/35862

§ 371 Date: Oct. 29, 2015

Related U.S. Application Data
(60) Provisional application No. 61/816,857, filed on Apr. 29, 2013.

(51) Int. Cl. A61K 8/92; A61Q 19/10; A61Q 5/02; A61Q 5/00; A61K 8/97; A61Q 19/00; A61Q 1/04; A61K 8/19; A61Q 9/02

(52) U.S. Cl. CPC A61K 8/922; A61K 8/19; A61Q 19/10; A61Q 5/02; A61Q 9/02; A61K 8/97; A61Q 19/001; A61Q 1/04; A61Q 5/006; A61Q 19/007; A61K 2800/30

(57) ABSTRACT

A soap composition for the treatment of skin conditions and irritations, including 40-50 wt % soy oil, 15-25 wt % coconut oil, 1-10 wt % olive oil, 0.5-5 wt % cocoa butter, 15-25 wt % water, and 5-15 wt % sodium hydroxide. A lotion composition, including 85-99 wt % lotion base, 2-10% neem oil, and 0.5-5% black seed oil. A lip balm composition, including 80-99.999 wt % lip balm base, and 0.001-20 wt % of a composition of castor oil, palm kernel oil, aloe extract, carnauba wax, candelilla wax, cocoa butter, vanilla oil, vanilla, and combinations thereof. A method of treating a skin irritation by applying the soap composition to skin, and relieving the irritation. A method of making a soap composition. A method of treating a skin irritation by applying the lotion composition to skin, and relieving the irritation.

### BACKGROUND OF THE INVENTION

1. Technical Field

The present invention relates to a composition of soap and methods of treating skin irritations.

2. Background Art

There are many different skin conditions that cause pain and irritation to individuals. Some skin conditions are chronic and must be managed by drugs and lifestyle changes, as they have no cure.

Eczema is a skin condition in which the skin is inflamed or irritated. Eczema can be associated with allergic conditions, such as asthma and hay fever. Symptoms include itchy skin and dry, thickened, or scaly skin. Lotions and creams are used to keep the skin moist and relieve itching. Hydrocortisone 1% cream or other creams with corticosteroids can be used. Antihistamines, tar treatments, and phototherapy are also prescribed. Colloidal oatmeal, 0.007% minimum, is a skin protectant that can be used in soaps. ELIDEL® (pimecrolimus, Meda Pharma S.A.R.L.) and PROTOPIC® (tacrolimus, Astellas) are used in extreme cases, but also have a cancer risk associated with use.

Rosacea is a skin condition that causes redness (flushing) and pimples on the nose, cheeks, chin, and forehead, as well as burning and soreness in the eyes and eyelids. Flushing can be caused by sun exposure, spicy foods, drinking hot beverages and alcohol, using sun protection, and using medication, and therefore, avoidance of these can reduce rosacea. Treatments include creams or gels, such as those with antibiotics of topical metronidazole, clindamycin, or erythromycin, azelaic acid or sodium sulfacetamide and sulfur, or tretinoin. Oral antibiotics can be prescribed. Isotretinoin can also be used but is discouraged because it causes birth defects.

Psoriasis is a skin condition that causes redness and irritation of the skin, common among many people. Symptoms of the condition are scales of flaky silver-white patches (dead skin cells) on red skin, most common at the elbows, knees, and mid-body. Severe dandruff can also be a symptom. Psoriasis is believed to be a genetic disease caused by an immune response mistaking healthy cells for dangerous substances and is not contagious. Many different things can trigger an attack of psoriasis, such as bacterial infections, viral infections, dry air, skin injuries, medicines, stress, lack of sunlight, sunburn, and too much alcohol. Several types of psoriasis exist, including erythrodermic (large areas of intense redness), guttate (small pink-red spots), inverse (redness and irritation in armpits, groin, and between overlapping skin), plaque (red skin with silver-white scales), and pustular (white blisters surrounded by red, irritated skin).

Current treatments of psoriasis include topical treatments (lotions, ointments, creams, and shampoos) that include ingredients such as cortisone, coal tar, anthralin, salicylic acid, lactic acid, vitamin D, or vitamin A. Systemic treatments are also used that affect the body's immune response in severe psoriasis, and include methotrexate, cyclosporine, or retinoids. Biologics are also used including adalimumab (HUMIRA®, Abbott Laboratories), alefacept (AMEVIVE®, Biogen, Inc.), etaneracept (ENBREL®, Amgen, Inc.), infliximab (REMICADE®, Janssen Biotech, Inc.), and stelara. Phototherapy can also be used with the application of ultraviolet A (UVA) or ultraviolet B (UVB) light to skin.

In general, the above treatments for skin conditions and irritations are not effective and can result in harmful side effects. There remains a need for a treatment of various skin conditions that is safe and effective.

### SUMMARY OF THE INVENTION

The present invention provides for a soap composition for the treatment of skin conditions and irritations, including 40-50 wt % soy oil, 15-25 wt % coconut oil, 1-10 wt % olive oil, 0.5-5 wt % cocoa butter, 15-25 wt % water, and 5-15 wt % sodium hydroxide.

The present invention provides for a lotion composition, including 85-99 wt % lotion base, 2-10% neem oil, and 0.5-5% black seed oil.

The present invention also provides for a lip balm composition, including 80-99.999 wt % lip balm base, and 0.001-20 wt % of a composition of castor oil, palm kernel oil, aloe extract, carnauba wax, candelilla wax, cocoa butter, vanilla oil, vanilla, and combinations thereof.

The present invention provides for a method of treating a skin irritation by applying the soap composition to skin, and relieving the irritation.

The present invention provides for a method of making a soap composition by pouring lye into cold water, stirring the lye and water mixture with a rubber spatula, heating the lye and water mixture and giving off gas, cooling the lye and water mixture to 92-105 degrees F., melting oils and fatty acids including soy oil, coconut oil, olive oil, and cocoa butter in a pot and cooling to 92-105 degrees F., mixing the lye and water mixture into the oil and fatty acid pot until optical inhomogeneities are present, pouring the mixture into a mold and preventing heat loss, forming soap removing the soap from the molds, and curing the soap.

The present invention further provides for a method of treating a skin irritation by applying the lotion composition to skin, and relieving the irritation.

### DETAILED DESCRIPTION OF THE INVENTION

The present invention is generally directed to a composition of soap for the treatment of skin conditions and irritations, as well as a lotion composition, and a lip balm composition.

The soap composition includes soy oil, coconut oil, olive oil, cocoa butter, water, and sodium hydroxide (commonly referred to as lye). The soap composition can optionally include at least 0.007 wt % colloidal oatmeal. The preferred amounts of each of these ingredients are listed in TABLE 1, along with ranges.

TABLE 1

| Ingredient | Preferred amount | Range |
|---|---|---|
| Soy oil | 55 oz | 40-50 wt % |
| Coconut oil | 25 oz | 15-25 wt % |
| Olive oil | 5 oz | 1-10 wt % |
| Cocoa butter | 2 oz | 0.5-5 wt % |
| Water | 24 oz | 15-25 wt % |
| Colloidal oatmeal | 0.9 oz | 0.007 wt % and greater |
| Sodium hydroxide | 12 oz | 5-15 wt % |

These ingredients can also be added to the lotion composition and lip balm composition as desired.

The soap composition can be in several different forms, such as, but not limited to, a bar soap, a liquid soap, shampoo, shaving soap, and body wash. The soap composition can be shaped by any as desired, such as a bar soap molds. The soap composition can be made any color desired or have any fragrance desired, and some examples are provided below. The soap composition is made by a saponification method known by those skilled in the art.

The soap composition is safe for use with human infants, children, and adults, as well as animals. However, the soap composition is not tearless.

Most preferably, the soap composition is used to treat skin conditions and irritations, such as, but not limited to, eczema, rosacea, acne, dandruff, allergic reactions, chicken pox, rashes, and psoriasis. The soap can be used to treat any skin condition in which the skin is red, itchy, dry, burning, or combinations thereof. Therefore, the present invention provides for a method of treating a skin irritation, by applying the soap composition to skin, and relieving the irritation. The relief can include making dry skin smooth and moist, returning skin to its normal color, stopping itching, unclogging pores, stopping burning and combinations thereof. The soap composition relieves the symptoms associated with skin conditions and irritations. Essentially, the soap composition returns the skin to a non-diseased state. The soap composition allows the user's skin to find its own natural pH. The soap composition cleans the skin and leaves no residues behind except for breathable properties to allow the skin to find balance and heal naturally without interference of animal fats and chemicals that clog pores.

For both lotions and soaps, the compositions of the present invention overcome problems of dry skin, eczema, psoriasis, and acne, and soothe itching experienced with past compositions. For the soap composition, about half of the improvement is due to process.

The soap composition can be made by the following cold process method. First, cold water is measured into a glass Pyrex or equivalent container. Lye (i.e. sodium hydroxide) is measured, and then slowly poured into the cold water container and the lye and water mixture is stirred with a Teflon or heat resistant rubber spatula. The mixture of the lye and water heats up and gives off gas. The mixture is set aside to cool to 92 to 105 degrees F., and preferably 97 to 99 degrees F. Oils and fatty acids (i.e. soy oil, coconut oil, olive oil, and cocoa butter described above) are measured into a large pot and heated on a burner at medium heat until all are melted. The oils and fatty acids are removed from heat and set aside to cool to 92 to 105 degrees F., and preferably 97 to 99 degrees F. The form/mold is prepared. With a high-speed mixer/emulsifier, the lye mixture is mixed (at a trickle) into the oil/fatty acid pot (only when both containers are at the desired temperatures). Once mixed, the mixture can continue to be pulsed/mixed off and on as needed until optical inhomogeneities in the mixture become evident/are present. The addition of colloidal oatmeal, when indicated, occurs after the admixture of the other soap ingredients. The mixture is poured into the prepared form/mold and the form/mold is then covered and wrapped with blankets or towels to prevent heat loss. After 12 to 48 hours, preferably 20 to 24 hours, the soap is removed from the form/mold and cut into bars if necessary. The soap bars are placed on a shelf and cured for 24 to 48 hours.

The present invention also provides for a lotion composition that is paraben free including a lotion base (85-99 wt % lotion base), neem oil (2-10%, preferably 8-10% (v/v)), and black seed oil (0.5 to 5%, preferably 2-5% (v/v)). The lotion base can be premixed and bought from commercial sources and preferably includes compositions such as water, caprylic/capric triglyceride, stearic acid, isopropyl myristate, cetyl alcohol, glyceryl stearate, PEG-100 stearate, Aloe barbadensis leaf juice, Helianthus annuus seed oil (sunflower), Camellia sinensis leaf extract, Linum usitatissimum seed extract, Punica granatum extract, Olea europaea fruit extract, Laminaria digitat extract, Morinda citrifolia extract, shea butter, Lavendula angustifolia extract, honey extract, macademia seed oil, avocado oil, cetearyl alcohol, glycerin, carbomer, triethanolamine, tetrasodium EDTA, and combinations thereof. JPS additives are the key; the lotion base must be thick and creamy, not oily. The lotion base can be shea butter, which is especially useful for children with extremely sensitive skin.

Additional ingredients in the lotion composition can also include water, caprylic/capric trislycoride, stearic acid, isopropyl myristate, cetyl alcohol, glyceryl stearate, PEG-100 stearate, Aloe barbadensis leaf juice, Helianthus annus, seed oil (sunflower), Camellia sinensis leaf extract, Linum usitatissimum seed extract, Punica granatum extract, Olea europaea fruit extract, Laminaria digitat extract, Morinda citrifolia extract, Butyrospermum parkii (shea butter), Lavendula angustifolia flower/extract, honey extract, macadamia seed oil, black seed oil, avocado oil, raspberry seed oil, cetearyl alcohol, ceteareth-20, glycerin, DMDM hydantoin, carboreen, triethanolamin, tetrasodium EDTA, and combinations thereof. These ingredients can be in an amount of 0.001 wt % to 20 wt % of the total composition and are added for fragrance and additional healing properties.

Most preferably, the lotion composition is used to treat skin conditions and irritations, such as, but not limited to, eczema, rosacea, acne, dandruff, allergic reactions, chicken pox, rashes, and psoriasis. The lotion composition can be used to treat any skin condition in which the skin is red, itchy, dry, burning, or combinations thereof. Therefore, the present invention provides for a method of treating a skin irritation, by applying the lotion composition to skin, and relieving the irritation. The relief can include making dry skin smooth and moist, returning skin to its normal color, stopping itching, unclogging pores, stopping burning, providing skin regeneration, and combinations thereof. The lotion composition relieves the symptoms associated with skin conditions and irritations. Essentially, the lotion composition returns the skin to a non-diseased state.

The present invention further provides for a lip balm composition including a lip balm base, and optional ingredients of castor oil, palm kernel oil, aloe extract, carnauba wax, candelilla wax, cocoa butter, vanilla oil, vanilla, and combinations thereof in an amount of 0.001 wt % to 20 wt % of the total composition. Preferably, the lip balm composition includes cocoa butter (1-10%, preferably 3-5% (v/v)), vanilla oil (0.5-4%, preferably 1-2% (v/v)), and 86-98.5% lip balm base. The lip balm base is preferably vegan. The lip balm base can be premixed and bought from commercial sources, and preferably includes compositions such as castor oil, palm kernel oil, avocado oil, coconut oil, aloe extract, carbauba wax, candilla wax, and combinations thereof. The lip balm base can be carnauba wax, and can also be shea butter. The lip balm composition can be formed in a lip balm mold in any shape desired. The lip balm composition can be any color desired, it can also impart color to the lips by a coloring composition, or have any fragrance desired, and some examples are provided below. The lip balm base can be used for chapped lips, and general moisturization.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

Royal Bar. At 97 degrees F., the ingredients were added after saponification:

25 oz. coconut oil

55 oz. soy oil

5 oz. olive oil

24 oz. (3 cups) water

12 oz. sodium hydroxide

4 tablespoons aloe

4 tablespoons warmed honey

2 oz. cocoa butter

### EXAMPLE 2

Chocolate Almond. Ingredients include:

25 oz. coconut oil

55 oz. soy oil

5 oz. olive oil

24 oz. (3 cups) water

12 oz. sodium hydroxide

2 oz. sweet almond oil

fragrance

1 oz. chocolate flavor

2 teaspoons cocoa

### EXAMPLE 3

Healing lotion. 4 oz. total. Ingredients include:

0.3 oz Neem oil max (OTC) 7.5%

3.7 oz. lotion base recipe

0.04 oz. raspberry fragrance

1 70 mm lid

1 70 mm 4 oz. frosted jar

1 shrink band

1 label

### EXAMPLE 4

Simple almond lotion. Ingredients include:

0.1 oz. sweet almond oil 2.5%

3.9 oz. lotion base recipe

0.2 oz. honey/almond fragrance

1 4 oz. BRPET 24 mm

1 size 24 disk top

1 shrink band

1 label

### EXAMPLE 5

Red raspberry lotion. Ingredients include:

3.8 oz. lotion base recipe

0.02 oz. raspberry seed oil

1 4 oz. 24 mm

1 size 24 disk top

1 shrink band

1 label

### EXAMPLE 6

Hunters Moon lotion. Ingredients:

4 oz. lotion base recipe

0.02 oz. oak leaves and acorn fragrance

1 PET 24 mm 4 oz.

1 size 24 disk top

1 shrink band

1 label

### EXAMPLE 7

Lilac lotion. Ingredients:

0.02 oz. fragrance

4 oz. lotion base recipe

### EXAMPLE 8

Neem lotion. Ingredients:

3.7 oz. lotion base recipe

0.3 oz. Neem (7.5%-10%)

1 mL raspberry zest or infused vanilla

### EXAMPLE 9

Lip balm tube. Ingredients:

vegan lip base

5-6% pure vanilla

1 label

1 twist tube

1 cap

1 shrink band

transfer pipettes

### EXAMPLE 10

Shaving soap

The shaving soap was tested and sold at Dowen's Barber Shop in Corunna for almost three months with positive response.

Master formula:

3 cups water

71.2 oz. coconut oil

14.2 oz. caster oil

3.6 oz. olive oil

14.3 oz. sodium hydoxide

97 degrees F. bath at saponification.

The soap was cut into 2.5 inch rounds for bowl and brush shaving.

Foaming hand soap and 1 oz. master soap recipe to 11 oz. H₂0.

Formula for shaving cream machines:

7 oz. master soap to 10 oz H₂0.

Concentrate formula (16 oz. bottle) to mix with water; makes 1 gallon of mix.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A soap composition for the treatment of skin conditions and irritations, comprising 40-50 wt % soy oil, 15-25 wt % coconut oil, 1-10 wt % olive oil, 0.5-5 wt % cocoa butter, 15-25 wt % water, and 5-15 wt % sodium hydroxide.

2. The soap composition of claim 1, wherein said soap composition is in a form chosen from the group consisting of bar soap, a liquid soap, shampoo, shaving soap, and body wash.

3. The soap composition of claim 1, wherein said soap composition further includes a fragrance.

4. The soap composition of claim 1, wherein said soap composition further includes at least 0.007 wt % colloidal oatmeal.

5. A lotion composition for the treatment of skin conditions and irritations, comprising 85-99 wt % lotion base, 2-10% neem oil, and 0.5-5% black seed oil

6. The lotion composition of claim 5, wherein said lotion base includes a composition chosen from the group consisting of water, caprylic/capric triglyceride, stearic acid, isopropyl myristate, cetyl alcohol, glyceryl stearate, PEG-100 stearate, Aloe barbadensis leaf juice, Helianthus annuus seed oil (sunflower), Camellia sinensis leaf extract, Linum usitatissimum seed extract, Punica granatum extract, Olea europaea fruit extract, Laminaria digitat extract, Morinda citrifolia extract, shea butter, Lavendula angustifolia extract, honey extract, macademia seed oil, avocado oil, cetearyl alcohol, glycerin, carbomer, triethanolamine, tetrasodium EDTA, and combinations thereof.

7. The lotion composition of claim 5, wherein said lotion base is shea butter.

8. The lotion composition of claim 5, wherein said lotion composition further includes 0.001-20 wt % of a composition chosen from the group consisting of water, caprylic/capric trislycoride, stearic acid, isopropyl myristate, cetyl alcohol, glyceryl stearate, PEG-100 stearate, Aloe barbadensis leaf juice, Helianthus annus, seed oil (sunflower), Camellia sinensis leaf extract, Linum usitatissimum seed extract, Punica granatum extract, Olea europaea fruit extract, Laminaria digitat extract, Morinda citrifolia extract, Butyrospermum parkii (shea butter), Lavendula angustifolia flower/extract, honey extract, macadamia seed oil, black seed oil, avocado oil, raspberry seed oil, cetearyl alcohol, ceteareth-20, glycerin, DMDM hydantoin, carboreen, triethanolamin, tetrasodium EDTA, and combinations thereof.

9. A lip balm composition for the treatment of skin conditions and irritations, comprising 80-99.999 wt % lip balm base, and 0.001-20 wt % of a composition chosen from the group consisting of castor oil, palm kernel oil, aloe extract, carnauba wax, candelilla wax, cocoa butter, vanilla oil, vanilla, and combinations thereof.

10. The lip balm composition of claim 9, wherein said lip balm composition is paraben free.

11. The lip balm composition of claim 9, wherein said lip balm composition is further defined as comprising 86-98.5 wt % lip balm base, 1-10 wt % cocoa butter, and 0.5-4 wt % vanilla oil.

12. The lip balm composition of claim 9, wherein said lip balm base includes a composition chosen from the group consisting of castor oil, palm kernel oil, avocado oil, coconut oil, aloe extract, carbauba wax, candilla wax, shea butter, and combinations thereof.

13. The lip balm composition of claim 9, wherein said lip balm composition is formed in a lip balm mold.

14. The lip balm composition of claim 9, wherein said lip balm composition further includes a composition chosen from the group consisting of a coloring composition and a fragrance.

15. A method of treating a skin irritation, including the steps of:
applying a soap composition comprising 40-50 wt % soy oil, 15-25 wt % coconut oil, 1-10 wt % olive oil, 0.5-5 wt % cocoa butter, 15-25 wt % water, and 5-15 wt % sodium hydroxide to skin; and
relieving the irritation.

16. The method of claim 15, wherein the soap composition further comprises at least 0.007 wt % colloidal oatmeal.

17. The method of claim 15, wherein the irritation is caused by a condition chosen from the group consisting of eczema, rosacea, acne, dandruff, allergic reactions, chicken pox, rashes, and psoriasis.

18. The method of claim 15, wherein the irritation is caused by the skin being in a condition chosen from the group consisting of red, itchy, dry, burning, or combinations thereof.

19. The method of claim 15, wherein said relieving step is further defined as a relief chosen from the group consisting of making dry skin smooth and moist, returning skin to its normal color, stopping itching, unclogging pores, stopping burning, and combinations thereof.

20. The method of claim 15, further including the step of returning the skin to a non-diseased state.

21. The method of claim 15, further including the step of allowing the skin to find its own natural pH.

22. A method of making a soap composition, including the steps of:
pouring lye into cold water;
stirring the lye and water mixture with a rubber spatula, heating the lye and water mixture and giving off gas;
cooling the lye and water mixture to 92-105 degrees F.;
melting oils and fatty acids including soy oil, coconut oil, olive oil, and cocoa butter in a pot and cooling to 92-105 degrees F.;
mixing the lye and water mixture into the oil and fatty acid pot until optical inhomogeneities are present;
pouring the mixture into a mold and preventing heat loss;
forming soap removing the soap from the molds; and
curing the soap.

23. A method of treating a skin irritation, including the steps of:
applying a lotion composition comprising 85-99 wt % lotion base, 2-10% neem oil, and 0.5-5% black seed oil to skin; and
relieving the irritation.

24. The method of claim 23, wherein the irritation is caused by a condition chosen from the group consisting of eczema, rosacea, acne, dandruff, allergic reactions, chicken pox, rashes, and psoriasis.

25. The method of claim 23, wherein the irritation is caused by the skin being in a condition chosen from the group consisting of red, itchy, dry, burning, or combinations thereof.

26. The method of claim 23, wherein said relieving step is further defined as a relief chosen from the group consisting of making dry skin smooth and moist, returning skin to its normal color, stopping itching, unclogging pores, stopping burning, providing skin regeneration, and combinations thereof.

27. The method of claim 23, further including the step of returning the skin to a non-diseased state.
